(19) United States
(12) Patent Application Publication
(10) Pub. No.: US 2022/0211912 A1
(43) Pub. Date: Jul. 7, 2022

(54) HYDROGEL RETINAL TAMPONADE AGENT

(71) Applicants: Sherif Soliman, Irvine, CA (US); Oussama Boundaoui, Bridgeview, IL (US); Hamzah Khalaf, Helotes, TX (US); Sidi A. Bencherif, East Boston, MA (US)

(72) Inventors: Sherif Soliman, Irvine, CA (US); Oussama Boundaoui, Bridgeview, IL (US); Hamzah Khalaf, Helotes, TX (US); Sidi A. Bencherif, East Boston, MA (US)

(21) Appl. No.: 17/595,139

(22) PCT Filed: May 9, 2020
(86) PCT No.: PCT/US2020/032277

§ 371 Date: Nov. 9, 2021

Related U.S. Application Data
(60) Provisional application No. 62/845,338, filed on May 9, 2019.

(51) Int. Cl. A61L 27/52; A61L 27/54; A61L 27/58; A61L 27/38; A61L 27/20; C08L 5/04

(52) U.S. Cl. CPC A61L 27/52; A61L 27/54; A61L 27/58; A61L 27/3804; A61L 27/20; A61L 2300/414; A61L 2400/06; A61L 2430/16; A61L 2300/216; A61L 2300/404; A61L 2300/41; C08L 5/04

(57) ABSTRACT

A hydrogel retinal tamponade agent is disclosed herein. The disclosed hydrogel tamponade agent acts as a temporary barrier to fluid flow into the subretinal space and obviates the need to use intraocular gases or silicone oil as a retinal tamponade agent in a retinal detachment repair procedure. The hydrogel tamponade agent remains in place after a permanent seal is achieved and is slowly resorbed. The hydrogel tamponade agent is preferably biocompatible.

### CROSS-REFERENCE TO RELATED APPLICATIONS

This application claims the benefit of and priority to U.S. Provisional Patent Application Ser. No. 62/845,338, filed on May 9, 2019, the entirety of which is hereby incorporated herein by reference.

### BACKGROUND

### Field of the Invention

The present disclosure relates to tamponade agents for use in retinal detachment repair procedures.

### Description of the Related Art

A retinal detachment typically occurs when one or more tears or holes in the retina allow fluid to enter the subretinal space. As fluid continues to egress into this space, the retinal detachment enlarges. This causes a symptomatic visual field cut, or loss of central vision if the macula—the center of the retina—is involved. If untreated, a rhegmatogenous retinal detachment (RRD) —a retinal detachment caused by a break or tear in the retina—may potentially lead to blindness.

Retinal detachments are one of the most common indications for vitreoretinal surgery. Rhegmatogenous retinal detachment incidence varies geographically, with an incidence reported between 6.3 and 17.9 per 100,000 people. See Mitry, D., et al. “The Epidemiology of Rhegmatogenous Retinal Detachment: Geographical Variation and Clinical Associations,” Br. J. Ophthalmol. 2010, 94(6), 678-84. In the United States, the incidence is about 12 per 100,000 people. See Id.

Several techniques are used to repair retinal detachments. The various techniques may be used in a medical office or an operating room, as required by the characteristics and complexity of the retinal detachment. Surgical repair involves removal of the vitreous (the transparent, colorless, gelatinous mass that fills the space in the eye between the lens and the retina), evacuation of the subretinal fluid, and the use of expansile gases or silicone oil as tamponade agents to prevent reformation of the detachment and further fluid flow into the subretinal space. See Vaziri, K., et al. “Tamponade in the Surgical Management of Retinal Detachment,” Clin Ophthalmol. 2016, 10, 471-76. A tamponade agent is a temporary plug or tent inserted tightly into a wound or orifice to arrest hemorrhage. Id. In retinal detachment repair surgery, use of a tamponade agent provides surface tension across retinal breaks to prevent further fluid flow into the subretinal space until a permanent seal is achieved. Id. The permanent seal is subsequently achieved through retinopexy—photocoagulation laser therapy or cryotherapy is used to create a scar that adheres the retina to the back wall of the eye. See Id.

Expansile gases have been used for over one hundred years in eye surgery, and are still considered to be the gold standard in vitreoretinal surgery. See Janco, L., et al. “Gases in Vitreoretinal Surgery,” Cesk. Slov. Oftalmol. 2012, 68(1), 3-8. The utility of expansile gases as tamponade agents have made them invaluable in retinal detachment repair, as well as in other procedures such as macular hole surgery. However, the use of expansile gases in retinal detachment repair still poses several problems both intraoperatively and postoperatively. These problems include: (1) intraocular hypertension caused by the effect of the gas on fluid flow within the eye; (2) the possibility of errors in diluting the gas intraoperatively, leading to incorrect gas concentrations, expanding gas, severely elevated intraocular pressure, and permanent vision loss; (3) requiring patients to maintain awkward head positions during placement of the tamponade effect in the area of the retinal break; (4) restrictions on patient air travel on account of rapid expansion of gases under decreased pressure that would lead to dangerously elevated intraocular pressures during flight, thereby creating problems for patients who are, for example, in remote areas, island residents, or deployed military personnel; (5) egress of gas into the subretinal space, which may prevent retinal reattachment or induce new retinal detachments; (6) hampering of postoperative fundus view and visual acuity testing; (7) poor patient vision for several weeks following surgery, which affects the patient's function and balance; (8) movement of gas to the anterior chamber, leading to pressure elevation and inflammation; and (9) increased incidence of visually significant cataract, which often requires extraction and further surgical intervention.

Silicone oil is another commonly used tamponade agent. However, the use of silicone oil requires a follow up surgery to remove the oil. Silicone oil also may egress into the subretinal space, which may prevent retinal reattachment or induce new retinal detachments. Silicone oil may also move into the anterior chamber, leading to pressure elevation and inflammation. In addition, silicone oil also leads to increased incidence of visually significant cataract, which often requires extraction and further surgical intervention.

Thus there remains an unmet need for a tamponade agent for use in retinal detachment repair procedures that overcomes the limitations of using expansile gases or silicone oil as the tamponade agent therein.

### SUMMARY

A hydrogel retinal tamponade agent is disclosed herein. The disclosed hydrogel tamponade agent acts as a temporary barrier to fluid flow into the subretinal space and obviates the need to use intraocular gases or silicone oil as a retinal tamponade agent in a retinal detachment repair procedure. The hydrogel tamponade agent remains in place after a permanent seal is achieved and is slowly resorbed. The hydrogel tamponade agent preferably is biocompatible.

Methods of using the disclosed hydrogel retinal tamponade agent in retinal detachment repair procedures are also disclosed herein. In some embodiments, the disclosed method includes the following steps in order:
(a) subretinal fluid is removed and a gas such as air is used to reattach the retina back to its anatomic location;
(b) a pre-hydrogel solution that forms a hydrogel or a hydrogel is injected at the site of the retinal tear, wherein the hydrogel is preferably bioadherant and temporarily fixates the retina to subretinal tissue to form a barrier for fluid flow into the subretinal space; and
(c) retinopexy is performed using standard laser or cryotherapy techniques to induce a permanent seal.
   Once the gel adheres, the gas may be replaced with fluid again to confirm successful barrier formation. Retinopexy results in formation of a scar that permanently seals the retina to underlying subretinal tissue. This prevents fluid egress subretinally and subsequent redetachment.

The hydrogel is preferably administered as a pre-hydrogel solution into the subretinal space, where the pre-hydrogel solution is composed of a pre-hydrogel in an appropriate solvent. The pre-hydrogel solution may preferably be administered by cannula using known techniques for the administration of retinal tamponade agents. The pre-hydrogel solution converts into a soft hydrogel within minutes after injection to replace the vitreous, fill in the space, and ultimately seal the unsealed space caused by the retinal detachment and allow permanent sealing by retinopexy.

The disclosed hydrogel may be generated by non-covalent chemical interactions (e.g., ionic bonding, hydrogen bonding, hydrophobicity or hydrophilicity, stereocomplexation, protein-protein interactions) or covalent chemical crosslinking (e.g., radical polymerization, click chemistry, Michael addition, polycondensation, aldehyde-induced crosslinking).

The disclosed hydrogel may be any suitable biocompatible hydrogel, such as an ionically crosslinked anionic polymer (e.g., polysaccharides such as alginates); an ionically crosslinked cationic polymer (e.g., chitosan, cationic guar, cationic starch, polyethylene amine); or a covalently crosslinked polymer, including biocompatible synthetic polymers (e.g., PEG) and naturally-derived polymers (e.g., polysaccharides such as hyaluronic acid, carboxymethyl-cellulose, chitosan, and aloe vera, proteins such as gelatin, collagen, laminin, and fibronectin, peptides).

In some embodiments, the disclosed hydrogel may be an ionically crosslinked anionic polymer. In some preferred embodiments, the disclosed hydrogel may be an alginate.

In some preferred embodiments, the disclosed hydrogel may be bioresorbant. In some embodiments, the disclosed hydrogel may be semi-opaque.

In some embodiments, the bioadhesive properties of the disclosed hydrogel may be enhanced by conjugation or grafting of the hydrogel or pre-hydrogel with one or more adhesive catecholic residues such as dopamine, PEGylated dopamine, L-DOPA, or resorcin. In alternative embodiments, the bioadhesive properties of the disclosed hydrogel may be enhanced by incorporating one or more adhesive catecholic residues such as dopamine, PEGylated dopamine, L-DOPA, or resorcin as co-monomers in the hydrogel or pre-hydrogel.

### BRIEF DESCRIPTION OF THE DRAWINGS

FIGS. 1A-1B show an illustration of an embodiment of the disclosed method of using a hydrogel tamponade agent in a retinal detachment repair procedure.

FIG. 2A shows the basic structure of an alginate and a method of forming a calcium alginate.

FIG. 2B shows an expanded view of the egg box model of a calcium alginate.

### DETAILED DESCRIPTION OF THE ILLUSTRATED EMBODIMENTS

A hydrogel retinal tamponade agent is disclosed herein. The disclosed hydrogel tamponade agent acts as a temporary barrier to fluid flow into the subretinal space and obviates the need to use intraocular gases or silicone oil as a retinal tamponade agent in a retinal detachment repair procedure. The hydrogel tamponade agent remains in place after a permanent seal is achieved and is slowly resorbed. The hydrogel tamponade agent preferably is biocompatible.

The term hydrogel as used in many references refers to both the gelled form and the pre-gelled form. As used herein, the term hydrogel will refer to the gelled form and the term pre-hydrogel will refer to the pre-gelation form. However, it will be understood by an ordinary skilled artisan that any agents described below that are described as incorporated into a hydrogel or any modifications of the hydrogel described below will be incorporated into or made to the pre-hydrogel that will then gel to form the hydrogel.

Methods of using the disclosed hydrogel retinal tamponade agent in retinal detachment repair procedures are also disclosed herein. In some embodiments, the disclosed method includes the following steps in order:
(a) subretinal fluid is removed and a gas such as air is used to reattach the retina back to its anatomic location;
(b) a pre-hydrogel solution that forms a hydrogel or a hydrogel is injected at the site of the retinal tear, wherein the hydrogel is preferably bioadherant and temporarily fixates the retina to subretinal tissue to form a barrier for fluid flow into the subretinal space; and
(c) retinopexy is performed using standard laser or cryotherapy techniques to induce a permanent seal.
   Once the gel adheres, the gas may be replaced with fluid again to confirm successful barrier formation. Retinopexy results in formation of a scar that permanently seals the retina to underlying subretinal tissue. This prevents fluid egress subretinally and subsequent redetachment.

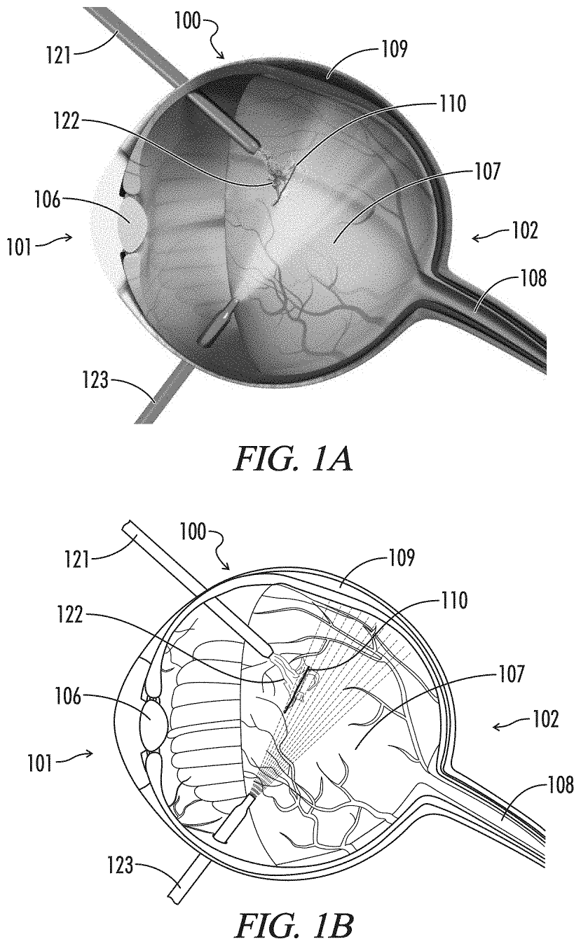

FIGS. 1A-1B show an illustration of an embodiment of the disclosed method of using a hydrogel tamponade agent in a retinal detachment repair procedure. An eye 100 with an anterior end 101 and a posterior end 102 that is exhibiting a rhegmatogenous retinal detachment is depicted. The eye 100 includes a lens 106, vitreous 107, and an optic nerve 108, and the eye 100 is exhibiting a retinal detachment 109 with a retinal tear 110. A cannula 121 is used to inject a pre-hydrogel solution 122 to form a tamponade at the location of the retinal tear 110. A light source 123 is used to illuminate the examination field.

There are numerous advantages of using the disclosed hydrogel technology in lieu of intraocular expansile gases for retinal detachment repair. These advantages include: (1) reduced incidence of postoperative complications such as cataracts and elevated intraocular pressure; (2) better postoperative vision in the absence of a gas; (3) enhanced postoperative comfort for the patient by obviating the need to maintain difficult head positions; (4) the absence of air travel restrictions, thereby allowing certain patients to return home sooner; and (5) no second surgery required as for silicone oil tamponade agents.

The hydrogel is preferably administered as a pre-hydrogel solution into the subretinal space, where the pre-hydrogel solution is composed of a pre-hydrogel in an appropriate solvent. The pre-hydrogel solution may preferably be administered by cannula using known techniques for the administration of retinal tamponade agents. The pre-hydrogel solution converts into a soft hydrogel within minutes after injection to replace the vitreous, fill in the space, and ultimately seal the unsealed space caused by the retinal detachment and allow permanent sealing by retinopexy.

The disclosed hydrogel may be generated by non-covalent chemical interactions (e.g., ionic bonding, hydrogen bonding, hydrophobicity or hydrophilicity, stereocomplexation, protein-protein interactions) or covalent chemical crosslinking (e.g., radical polymerization, click chemistry, Michael addition, polycondensation, aldehyde-induced crosslinking).

The disclosed hydrogel may be any suitable biocompatible hydrogel, such as an ionically crosslinked anionic polymer (e.g., polysaccharides such as alginates); an ionically crosslinked cationic polymer (e.g., chitosan, cationic guar, cationic starch, polyethylene amine); or a covalently crosslinked polymer, including biocompatible synthetic polymers (e.g., PEG) and naturally-derived polymers (e.g., polysaccharides such as hyaluronic acid, carboxymethyl-cellulose, chitosan, and aloe vera, proteins such as gelatin, collagen, laminin, and fibronectin, peptides).

In some embodiments, the disclosed hydrogel may be an ionically crosslinked anionic polymer. In some preferred embodiments, the disclosed hydrogel may be an alginate.

In some preferred embodiments, the disclosed hydrogel may be bioresorbant. In some embodiments, the disclosed hydrogel may be semi-opaque.

In some embodiments, the bioadhesive properties of the disclosed hydrogel may be enhanced by conjugation or grafting with one or more adhesive catecholic residues such as dopamine, PEGylated dopamine, L-DOPA, or resorcin. In alternative embodiments, the bioadhesive properties of the disclosed hydrogels may be enhanced by incorporating one or more adhesive catecholic residues such as dopamine, PEGylated dopamine, L-DOPA, or resorcin as co-monomers.

### Hydrogel Preparation

In some preferred embodiments, the disclosed hydrogel may be an alginate. In some highly preferred embodiments, the disclosed hydrogel may be a calcium alginate.

Alginate is an anionic copolymer composed of homopolymeric regions of 1,4-linked β-D-mannuronic acid (M-blocks) and α-L-guluronic acid (G-blocks) interspersed with regions of alternating structure. Gelation occurs when divalent cations (e.g., Ca²⁺, Ba²⁺, Fe²⁺, Sr²⁺) or trivalent cations (e.g., Al³⁺) are involved in interchain ionic bonding between G-blocks in the polymer chain, giving rise to a three-dimensional network. Such binding zones between the G-blocks are often referred to as “egg boxes.” These ions act as crosslinkers that stabilize alginate chains and form a gel structure containing crosslinked chains interspersed with more freely movable chains that bind and entrap large quantities of water. The gelation process is characterized by reorganization of the gel network accompanied by the expulsion of water. Alginate is non-toxic and biocompatible, and it is widely used in the medical, pharmaceutical, cosmetic, and food industries.

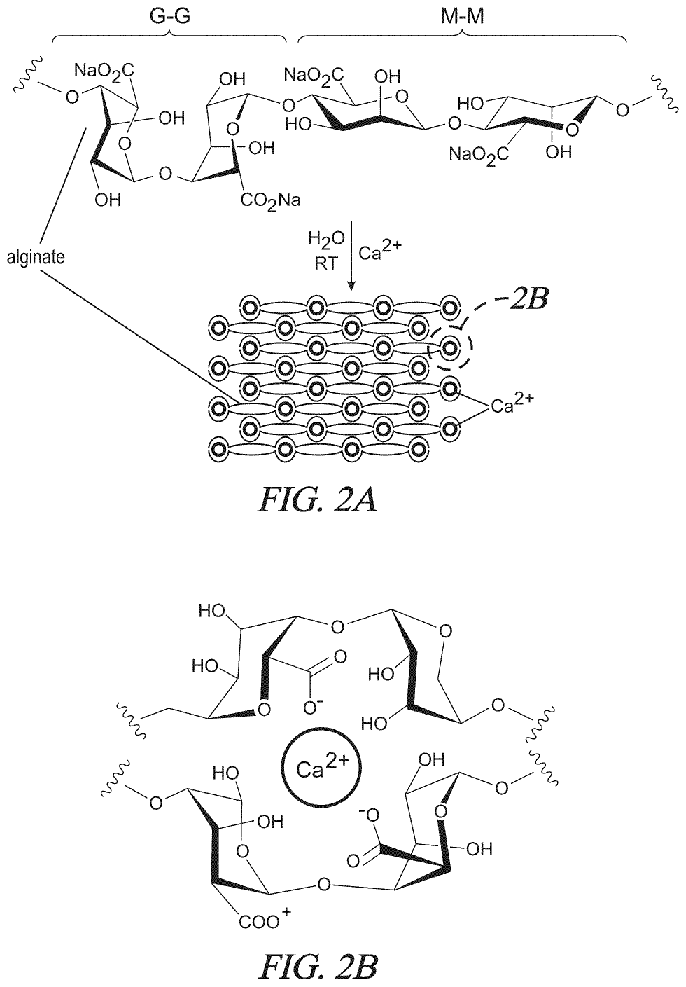

FIG. 2A shows the basic structure of an alginate, depicting a G-block and an M-block, and a method of forming a calcium alginate. FIG. 2B shows an expanded view of the egg box model of a calcium alginate.

The properties of the disclosed hydrogel, including syringe injectability, gelation time, swelling, mechanics, and bioadhesion, may be optimized by fine-tuning various parameters. For example, for a calcium alginate hydrogel these parameters include polymer concentration in the pre-hydrogel solution, the type of alginate used, the extent of alginate partial oxidation, calcium concentration, the identity of the calcium salt used, and the incorporation of biomolecules or lack thereof into the alginate. Other hydrogels may be optimized for the desired application by fine-tuning some or all of the same parameters or analogous parameters specific to the hydrogel used. The pre-hydrogel solution may be injected at various volumes as low as a few microliters while enabling gelation under physiological conditions, namely a temperature of 37° C. and a pH of 7.4.

In some preferred embodiments, the disclosed alginate hydrogel may be calcium crosslinked. In alternate embodiments, the disclosed alginate hydrogel may be ionically crosslinked with magnesium or potassium. In other alternate embodiments, the disclosed alginate hydrogel may be covalently crosslinked using orthogonal click reactions (e.g., click chemistry), Michael addition, or other suitable crosslinking reactions.

The pre-hydrogel solution may be crosslinked using metal salt solutions, such as calcium, magnesium, or potassium salt solutions. In some embodiments, the concentration of metal cation in the metal salt solution may preferably be between about 0.05 M and about 0.5 M.

In some preferred embodiments, the disclosed calcium alginate may be crosslinked using a solution of calcium sulfate (CaSO₄) and calcium carbonate D-gluconic acid lactone (CaCO₃-GDL). Calcium sulfate is fast-gelling and calcium carbonate D-gluconic acid lactone is slow-gelling. The absolute and relative concentrations of CaSO₄ and CaCO₃-GDL may be selected to optimize the desired properties of the hydrogel. The time-delayed release of crosslinking calcium cations allows the calcium alginate suspension to be injected before gelation occurs. In some embodiments, the gelation time is preferably between about 1 min and 20 mins. In alternate embodiments, other calcium salts and their related hydrates (e.g., CaCl₂, CaSO₄.0.5H₂O) may be used.

The concentration of alginate solution and the molecular weight of the alginate may preferably be selected to optimize the properties of the hydrogel (e.g., mechanics, swelling). The alginate solution may preferably be between about 0.5% and 10% (weight/volume) and the molecular weight of the alginate may be between about 10 kDa and 2000 kDa.

Aliginate hydrogels typically degrade in a predictable manner, which is an important structural feature that allows the controlled release of therapeutic biomolecules or cells into damaged tissues and align the gel degradation rate with the tissue regeneration rate. Alginate is not naturally enzymatically degraded in mammals, and thus ionically crosslinked alginate hydrogels exhibit a remarkably slow degradation rate in vivo, typically between months and years for the complete removal of an alginate hydrogel from an injection site. In some preferred embodiments, the degradation kinetics of the disclosed alginate hydrogel may be controlled by varying alginate molecular weight or by incorporating biodegradable crosslinks. In other preferred embodiments, the disclosed alginate hydrogel may be oxidized to control the degradation kinetics thereof. Oxidization generally accelerates the degradation rate of an alginate hydrogel. When alginate is oxidized by reaction with sodium periodate, the carbon-carbon bonds of the cis-diol groups in the uronate residues are cleaved and converted into dialdehyde groups. This approach allows control over the degradation rate by varying the degree of oxidation—increasing the degree of oxidation increases the vulnerability of alginate hydrogels to hydrolysis. In some preferred embodiments, the alginate may be oxidized with sodium periodate to achieve a wide range of alginate oxidation, such as between about 1% and about 80% oxidation. In alternate embodiments, other oxidizing agents such as potassium persulfate, ammonium persulfate, or hydrogen peroxide may be used to oxidize the alginate.

In some embodiments, the bioadhesive properties of the disclosed hydrogel may be enhanced by conjugation or grafting with one or more adhesive catecholic residues such as dopamine, PEGylated dopamine, L-DOPA, or resorcin. In alternative embodiments, the bioadhesive properties of the disclosed hydrogels may be enhanced by incorporating one or more adhesive catecholic residues such as dopamine, PEGylated dopamine, L-DOPA, or resorcin as co-monomers. In some preferred embodiments, the adhesive catecholic residue is dopamine. Dopamine may preferably be conjugated with alginate at a grafting density of about 1-30% dopamine per repeat unit to form an injectable tissue adhesive hydrogel. A tissue adhesive dopamine-containing alginate hydrogel is expected to remain in place at the injection site and adhere to surrounding tissues. Tissue adhesiveness enables integration with the surrounding tissue following gel injection at the targeted site.

In some embodiments, a secondary therapeutic agent may be incorporated into the hydrogel. The secondary therapeutic agent may, for example, be one or more of anti-inflammatory drugs, antimicrobial agents, growth factors to promote local tissue regeneration, retinal progenitor cells, or other suitable secondary therapeutic agents.

### Permeability Testing

An effective tamponade agent must be impermeable to fluid. To demonstrate that the disclosed hydrogel tamponade agents are impermeable to fluid under the conditions encountered during retinal detachment repair procedures, a bovine eye was obtained. A 2 mm hole was made in the eye and a pre-hydrogel solution was injected into the hole. The pre-hydrogel solution was allowed to form a hydrogel over 5 minutes. After 5 minutes post-injection, a saline solution was poured over the hydrogel that formed. None of the saline solution was observed leaking or otherwise permeating through the hydrogel. Thus the hydrogel was deemed to be impermeable to the saline solution and suitable for use as a tamponade agent.

The previous description of the disclosed embodiments is provided to enable any person skilled in the art to make or use the invention disclosed herein. Although the various inventive aspects are disclosed in the context of certain illustrated embodiments, implementations, and examples, it should be understood by those skilled in the art that the invention extends beyond the specifically disclosed embodiments to other alternative embodiments and/or uses of the invention and obvious modifications and equivalents thereof. In addition, while a number of variations of various inventive aspects have been shown and described in detail, other modifications that are within their scope will be readily apparent to those skilled in the art based upon reviewing this disclosure. It should be also understood that the scope of this disclosure includes the various combinations or sub-combinations of the specific features and aspects of the embodiments disclosed herein, such that the various features, modes of implementation, and aspects of the disclosed subject matter may be combined with or substituted for one another. The generic principles defined herein may be applied to other embodiments without departing from the spirit or scope of the disclosure. Thus, the present disclosure is not intended to be limited to the embodiments shown herein but is to be accorded the widest scope consistent with the principles and novel features disclosed herein.

Similarly, the disclosure is not to be interpreted as reflecting an intent that any claim set forth below requires more features than are expressly recited in that claim. Rather, as the following claims reflect, inventive aspects may reside in a combination of fewer than all features of any single foregoing disclosed embodiment.

Each of the foregoing and various aspects, together with those set forth in the claims and summarized above or otherwise disclosed herein, including the figures, may be combined without limitation to form claims for a device, apparatus, system, method of manufacture, and/or method of use.

All references cited herein are hereby expressly incorporated by reference.

What is claimed is:

1. A method of repairing a retinal detachment comprising the following steps in order:
a. removing a subretinal fluid from a retina;
b. reattaching the retina using a gas;
c. injecting a hydrogel-forming pre-hydrogel tamponade agent or a hydrogel tamponade agent at a site of a retinal tear; and
d. performing a retinopexy procedure.

2. The method of claim 1, wherein the hydrogel-forming pre-hydrogel tamponade agent or hydrogel tamponade agent is bioadherent.

3. The method of claim 1, wherein the hydrogel-forming pre-hydrogel tamponade agent or hydrogel tamponade agent is a hydrogel-forming pre-hydrogel tamponade agent and is injected as a pre-hydrogel solution comprising a pre-hydrogel in a solvent.

4. The method of claim 3, wherein the pre-hydrogel solution forms a hydrogel selected from the group consisting of an ionically crosslinked anionic polymer, an ionically crosslinked cationic polymer, a covalently crosslinked polymer, and a naturally-derived polymer.

5. The method of claim 4, wherein the pre-hydrogel solution forms a hydrogel that is an ionically crosslinked anionic polymer.

6. The method of claim 5, wherein the ionically crosslinked anionic polymer is an alginate.

7. The method of claim 6, wherein the alginate is a calcium alginate.

8. The method of claim 6, wherein the alginate is a magnesium alginate.

9. The method of claim 6, wherein the alginate is a potassium alginate.

10. The method of claim 6, wherein the alginate is a covalently crosslinked alginate.

11. The method of claim 4, wherein the hydrogel is bioresorbant.

12. The method of claim 7, wherein the hydrogel is bioresorbant.

13. The method of claim 4, wherein the hydrogel is semi-opaque.

14. The method of claim 4, wherein the bioadhesive properties of the hydrogel are enhanced by conjugation or grafting the pre-hydrogel with one or more adhesive catecholic residues.

15. The method of claim 14, wherein the one or more adhesive catecholic residues is selected from the group consisting of dopamine, PEGylated dopamine, L-DOPA, and resorcin.

16. The method of claim 14, wherein the adhesive catecholic residue is dopamine.

17. The method of claim 16, wherein the dopamine is conjugated with the alginate at a grafting density of about 1-30% dopamine per alginate repeat unit.

18. The method of claim 4, wherein the bioadhesive properties of the hydrogel are enhanced by incorporating one or more adhesive catecholic residues into the pre-hydrogel as a co-monomer.

19. The method of claim 18, wherein the one or more adhesive catecholic residues is selected from the group consisting of dopamine, PEGylated dopamine, L-DOPA, and resorcin.

20. The method of claim 18, wherein the adhesive catecholic residue is dopamine.

21. The method of claim 3, wherein the pre-hydrogel solution is capable of gelation under physiological conditions.

22. The method of claim 4, wherein the pre-hydrogel solution is capable of gelation under physiological conditions.

23. The method of claim 6, wherein the alginate is formed using a pre-alginate solution that is between about 0.5% weight/volume and 10% weight/volume.

24. The method of claim 6, wherein the molecular weight of the alginate is between about 10 kDa and 2000 kDa.

25. The method of claim 6, wherein the alginate is oxidized by reaction with an oxidizing agent when the alginate is in pre-hydrogel form.

26. The method of claim 25, wherein the oxidizing agent is selected from the group consisting of sodium periodate, potassium persulfate, ammonium persulfate, and hydrogen peroxide.

27. The method of claim 1, wherein one or more therapeutic agents is incorporated into the hydrogel tamponade agent or pre-hydrogel tamponade agent.

28. The method of claim 27, wherein the therapeutic agent is one or more therapeutic agents selected from the group consisting of anti-inflammatory drugs, antimicrobial agents, growth factors to promote local tissue regeneration, and retinal progenitor cells.

29. The method of claim 3, wherein one or more therapeutic agents is incorporated into the pre-hydrogel.

30. The method of claim 29, wherein the therapeutic agent is one or more therapeutic agents selected from the group consisting of anti-inflammatory drugs, antimicrobial agents, growth factors to promote local tissue regeneration, and retinal progenitor cells.

31. The method of claim 4, wherein one or more therapeutic agents is incorporated into the pre-hydrogel.

32. The method of claim 31, wherein the therapeutic agent is one or more therapeutic agents selected from the group consisting of anti-inflammatory drugs, antimicrobial agents, growth factors to promote local tissue regeneration, and retinal progenitor cells.

33. The method of claim 5, wherein one or more therapeutic agents is incorporated into the pre-hydrogel.

34. The method of claim 33, wherein the therapeutic agent is one or more therapeutic agents selected from the group consisting of anti-inflammatory drugs, antimicrobial agents, growth factors to promote local tissue regeneration, and retinal progenitor cells.

35. The method of claim 6, wherein one or more therapeutic agents is incorporated into the pre-hydrogel.

36. The method of claim 35, wherein the therapeutic agent is one or more therapeutic agents selected from the group consisting of anti-inflammatory drugs, antimicrobial agents, growth factors to promote local tissue regeneration, and retinal progenitor cells.

37. The method of claim 7, wherein one or more therapeutic agents is incorporated into the pre-hydrogel.

38. The method of claim 37, wherein the therapeutic agent is one or more therapeutic agents selected from the group consisting of anti-inflammatory drugs, antimicrobial agents, growth factors to promote local tissue regeneration, and retinal progenitor cells.

39. The method of claim 1 further comprising the step replacing the gas with a fluid after injecting the hydrogel tamponade agent and before performing the retinopexy procedure.
